(19) United States
(12) Patent Application Publication
(10) Pub. No.: US 2012/0234321 A1
(43) Pub. Date: Sep. 20, 2012

(54) AEROSOLISATION SYSTEM

(76) Inventors: John Sylvester Power, Moycullen (IE); Keith Gibbons, Doughiska (IE); Conor Paul Duffy, Roscahill (IE); Niall Smith, Alloa (GB); Dermot Clancy, Manorhamilton (IE)

(21) Appl. No.: 13/356,372

(22) Filed: Jan. 23, 2012

Related U.S. Application Data
(63) Continuation of application No. 12/412,799, filed on Mar. 27, 2009, now abandoned.
Application No. 12/412,799 is a continuation-in-part of application No. 12/058,255, filed on Mar. 28, 2008, now abandoned.
Application No. 12/412,799 is a continuation-in-part of application No. 12/058,304, filed on Mar. 28, 2008, now abandoned.
(60) Provisional application No. 60/907,311, filed on Mar. 28, 2007.
(60) Provisional application No. 61/073,582, filed on Jun. 18, 2008.
(60) Provisional application No. 61/100,510, filed on Sep. 26, 2008.
(60) Provisional application No. 61/100,515, filed on Sep. 26, 2008.

(51) Int. Cl. A61M 16/16

(52) U.S. Cl. 128/203.12

(57) ABSTRACT

The invention provides a combination of a micro pump 27 or a micro valve with a vibrating mesh nebuliser 2. This is powered by a controller 3. The controller 3 may have modifications to provide the electrical drive mechanism for the pump 27 in addition to fulfilling the aerosol/nebuliser drive requirements. In one case the system is used for humidifying gas in a ventilator circuit. A humidifying agent (sterile water or sterile saline) is aerosolised and then delivered to a ventilator circuit 100 coupled to the respiratory system of a patient.

### CROSS-REFERENCES TO RELATED APPLICATIONS

The present application is a continuation-in-part of U.S. patent application Ser. No. 12/058,255 filed Mar. 28, 2008 which claims the benefit of U.S. provisional Application No. 60/907,311 filed Mar. 28, 2007 and also a continuation-in-part of U.S. patent application Ser. No. 12/058,304 filed Mar. 28, 2008 which claims the benefit of U.S. provisional Application No. 60/907,311 filed Mar. 28, 2007. The present application also claims the benefit of U.S. provisional application No. 61/073,582 filed Jun. 18, 2008; U.S. provisional application No. 61/100,510 filed Sep. 26, 2008; and U.S. provisional application No. 61/100,515 filed Sep. 26, 2008

The complete disclosures of all of these are incorporated herein by reference.

### BACKGROUND OF THE INVENTION

The invention relates to aerosol delivery control and feed systems.

Aerosol output of existing vibrating mesh technologies is inherently variable between devices. If input power characteristics are constant the output between devices of the same type will still vary dependent upon several factors including drive frequency relationship to natural frequency and the aperture hole size range. There are no integrated systems where the delivery of the liquid to the vibrating mesh is finely controlled at a pre-determined rate. Gravity feed does not have the required flow accuracy and coupling to external infusion pumps is expensive and cumbersome.

There is therefore a need for a system which will address at least some of these issues.

### BRIEF SUMMARY OF THE INVENTION

According to the invention there is provided an aerosolisation system comprising an aerosol generator and a flow controlling device for delivery of fluid to be aerosolised to the aerosol generator.

In one embodiment the flow controlling device comprises a micropump. The micropump may comprise a diaphragm pump. The diaphragm pump may be driven by piezo activation.

In another embodiment the flow controlling device comprises a microvalve. In one case the valve is a solenoid valve.

In one embodiment the aerosol generator comprises a vibratable member having a plurality of apertures extending between a first surface and a second surface thereof. The first surface may be adapted to receive fluid to be aerosolised. The aerosol generator may be configured to generate an aerosol at the second surface. In one case the vibratable member is dome-shaped in geometry. The vibratable member may comprise a piezoelectric element.

In one embodiment the apertures in the vibratable member are sized to aerosolise fluid by ejecting droplets of the water such that the majority of the droplets by mass have a size of less than 5 micrometers.

In one case the system comprises a controller for controlling the operation of the aerosol generator and the flow controlling device. The controller is configured to control the pulse rate at a set frequency of vibration of the vibratable member. In one case the controller may be impedance matched to the aerosol generator.

In one embodiment the apparatus comprises means to determine whether fluid is in contact with the aerosol generator. The determining means may be configured to determine at least one electrical characteristic of the aerosol generator. The determining means may be configured to determine at least one electrical characteristic of the aerosol generator over a range of vibration frequencies. The determining means may be configured to compare the at least one electrical characteristic against a pre-defined set of data.

The invention also provides a ventilator circuit comprising a system of the invention.

### BRIEF DESCRIPTION OF THE DRAWINGS

The invention will be more clearly understood from the following description of some embodiments thereof, given by way of example only, with reference to the accompanying drawings, in which:—

FIG. 1A is a diagram of a delivery system according to the invention;

FIG. 1B is a perspective view of an apparatus for humidifying gas in a ventilator circuit according to the invention;

FIG. 2 is a schematic illustration of a part of an apparatus according to the invention;

FIG. 3 is a schematic illustration of a part of the apparatus of FIG. 1;

FIG. 4 is an exploded isometric view of an aerosol generator used in the invention;

FIG. 5 is a cross-sectional view of the assembled aerosol generator of FIG. 4;

FIG. 6 is a perspective view of a controller housing used in the apparatus of the invention;

FIGS. 7(a) and 7(b) are graphs of DC voltage versus time and AC voltage versus time respectively to achieve a 100% aerosol output;

FIGS. 8(a) and 8(b) are graphs of DC voltage versus time and AC voltage versus time respectively to achieve a 50% aerosol output—FIG. 8(a) illustrates the waveform output from a microprocessor to a drive circuit and FIG. 8(b) illustrates the waveform output from a drive circuit to a nebuliser;

FIGS. 9(a) and 9(b) are graphs of DC voltage versus time and AC voltage versus time respectively to achieve a 25% aerosol output—FIG. 9(a) illustrates the waveform output from a microprocessor to a drive circuit and FIG. 9(b) illustrates the waveform output from a drive circuit to a nebuliser;

FIG. 10 is a graph of AC voltage versus time; and illustrates an output waveform from a drive circuit to a nebuliser;

FIG. 11 is a graph of frequency versus current for another apparatus according to the invention;

FIG. 12 is a perspective view of another apparatus of the invention;

FIG. 13 is a cross sectional view of another apparatus according to the invention;

FIG. 14 is a perspective view of an apparatus according to the invention for use in a procedure involving insufflation of a body cavity, such as laparoscopic surgery;

FIG. 15 is a view similar to FIG. 14 of another apparatus of the invention; and

FIG. 16 is a view similar to FIG. 14 of a further apparatus of the invention.

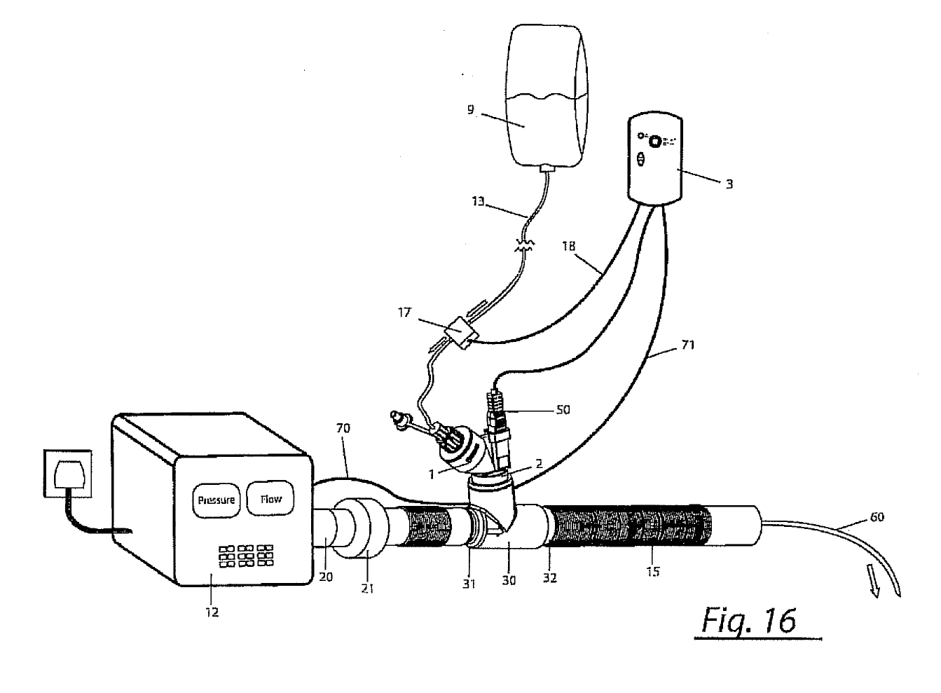

### DETAILED DESCRIPTION

The invention provides a combination of a micro pump with a vibrating mesh nebuliser. This is powered by a controller. The controller may have modifications to provide the electrical drive mechanism for the pump in addition to fulfilling the aerosol/nebuliser drive requirements.

The vibrating mesh aerosol generator can work with many types of micro pumps. Flow rates of pumps depend on the application and aerosol output requirements however they are typically in the range of 50 nano litres per minute to 5 millilitres per minute. Such micro pumps can have different means of providing the pumping action and can include membrane pumps, electrohydrodynamic (EHD) pumps, electrokinetic, (EK) pumps, rotary pumps, peristaltic pumps, phase change pumps, and several other types of pumps. Diaphragm pumps that are driven by piezo activation are of particular interest as much of the control circuitry utilised is similar to that used to drive vibrating mesh technology and therefore integration of the circuits is simpler and cheaper to undertake.

The invention also envisages the use of a micro valve with a vibrating mesh nebuliser powered by a controller. The controller may have modifications to provide the electrical drive mechanism for the valve in addition to fulfilling the aerosol/nebuliser drive requirements.

The vibrating mesh aerosol generator can work with many types of micro valves. Flow rates of valves depends on the application and aerosol output requirements however they are typically in the range of 50 nano litres per minute to 5 millilitres per minute. Such micro valves can have different means of providing the pumping action and can include solenoid valves, actuator valves. The use of a micro valve is direction dependent and requires a gravity or pressurized feed system.

The invention allows for the nebulization of liquids with surface tensions lower than water. These solutions are nebulizable but due to the surface tension they leak through the aperture plate when left sitting on it. When dispensed onto the aperture plate in a controlled drop by drop fashion the issue of leakage through the aperture plate will not occur.

The device also facilitates nebulisation of solutions that are prone to frothing. The potential to dispense the solution onto the aperture plate in a controlled fashion will prevent the build up of the solution on the aperture plate and the tendency to froth will be eliminated.

The device will reduce unnecessary exposure of solutions that are prone to oxidation or that are light sensitive.

As the pump feed can be located directly behind the aerosol plate it will remove the current restriction of the liquid feed being gravity dependent and will allow the creation of aerosol through 360° orientation of the device.

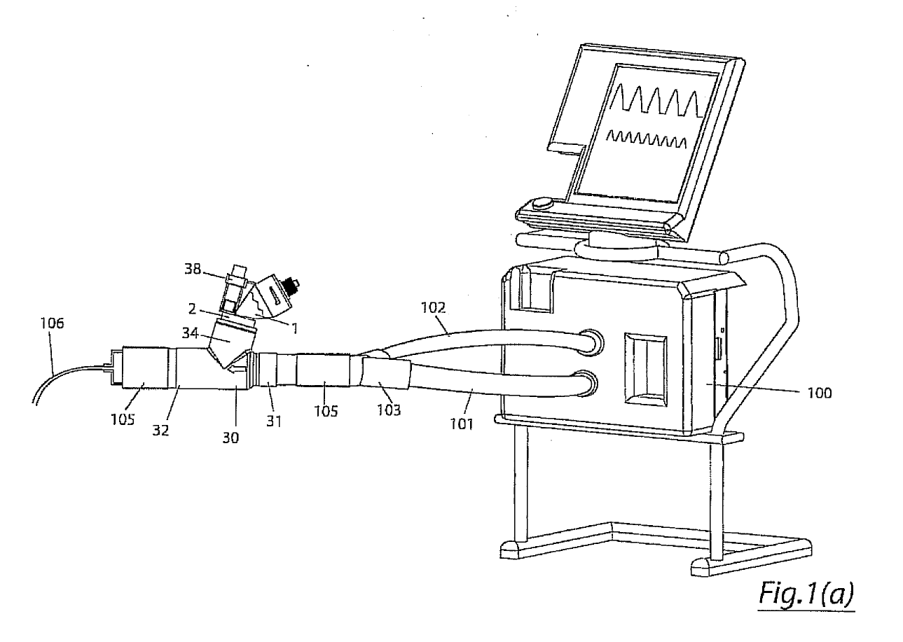

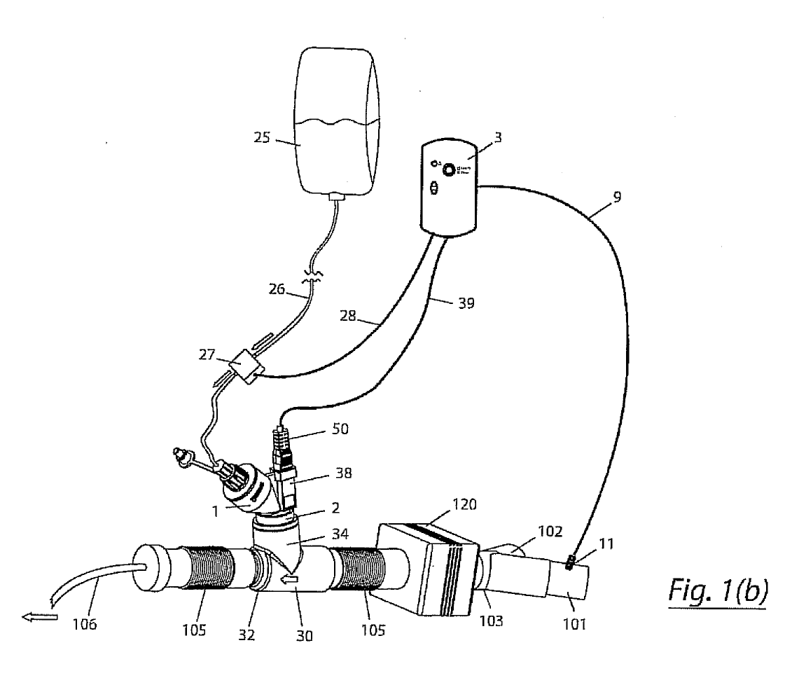

Referring to FIGS. 1 to 14 in one case the system is used for humidifying gas in a ventilator circuit. In the invention a humidifying agent (sterile water or sterile saline) is aerosolised and then delivered to a ventilator circuit coupled to the respiratory system of a patient.

The humidifying system of the invention is particularly useful in delivering the aerosolised humidifying agent to a patient whose breathing is being assisted by a ventilator 100 as illustrated diagrammatically in FIG. 1A. An inhalation or inspiration line 101 extends from the ventilator 100. A return or exhalation line 102 also extends to the ventilator 100. The inspiration and exhalation lines are connected to a junction piece 103, which may be a wye junction. A patient line 105 extends from the wye 103 to an endotracheal tube 106 which extends to the patients lungs. Generally, the various lines 101, 102, 105, 106 are provided by lengths of plastic tubing which are interconnected. The tubing defines lumens for passage of ventilation air, during the inspiration phase, along the inspiration line 101, patient lines 105 and endotracheal tubes 106 into the patients lungs. During the expiration phase exhaled air is delivered along the endotracheal tube 106, patient lines 105 and the expiration line 102. The wye junction 103 provides a common pathway for inspiration and exhalation between the junction 103 and the patients lungs. The ventilator 100 mechanically assists the flow of oxygenated air to the patient during the inspiration phase and in the exhalation phase a patient exhales, either naturally or by the ventilator applying negative pressure.

The apparatus comprises a reservoir 1 for storing sterile water or saline solution, the aerosol generator 2 for aerosolising the water, and a controller 3 for controlling the operation of the aerosol generator 2.

In one aspect of the invention, an aerosol generator 2 is used to deliver an aerosolised humidifying agent into the ventilation air during the inspiration phase.

In the arrangement of FIG. 1B the apparatus also comprises a sensor 11 for determining flow of air in the inspiration line 10. The sensor 11 is connected by a control wire 9 to the controller 3, and the aerosol generator 2 is also connected to the controller 3.

The humidifying agent may be sterile water or sterile saline with a salt concentration in the range from 1 micromolar to 154 millimolar. Such saline concentrations can be readily nebulised using the aerosolisation technology used in the invention.

In the invention an aerosol is delivered into the breathing circuit. The distinction between aerosol and vapour is in the size of the particles. The majority of aerosol particles that the aerosol generator produces are in the 0.5 to 5.0 micron diameter range. Water vapour on the other hand contains individual water molecules which are approximately 0.00001 microns i.e. 10,000 times smaller than the aerosol particles.

In the invention medical gases for those patients on mechanical ventilation are humidified. The lung is conditioned to receive gas at close to 100% relative humidity (RH). In the invention when undergoing mechanical ventilation the gas is also at 100% RH when exiting the endotracheal tube.

The amount of water a gas can hold is directly proportional to the temperature of the gas. The table below demonstrates the amount of water that air can hold at various temperatures to give 100% relative humidity

| | Amount of H₂O required per L to give |
|---|---|
| Air Temperature ° C. | 100% RH |
| 10 | 9.4 mg |
| 20 | 17.4 mg |
| 30 | 30.5 mg |
| 37 | 44.1 mg |

Thus, adding 0.044 ml of H₂O to 1 L of dry air at 37° C. will result in the air having a relative humidity of 100%, making it suitable for patients undergoing mechanical ventilation.

This aerosol generator 2 converts the water into an aerosol of a very definable particle size. The volume mean diameter (vmd) would typically be in the range of 2-10 microns.

The controller 3 is used to provide electrical power to drive the aerosol generator. This provides the aerosolising action to convey humidification to the breathing circuit.

Referring to FIGS. 1A and 1B, in this case an aerosol generator is placed between the Wye 103 and the endotracheal tube (ET) 106 to the patient. The aerosol generator is used to generate an aerosol of sterile water or sterile saline to humidify the gas being delivered to the patient.

In the arrangement of FIG. 1B 100% humidity at the end of the ET tube is achieved by having the nebulizer separate from the HME acting to top-up (boost) the humidity that is lost when using passive humidification via a HME.

In the case of an HME booster the aerosol generator 2 is placed between the ET and a HME 120 as illustrated in FIG. 1B.

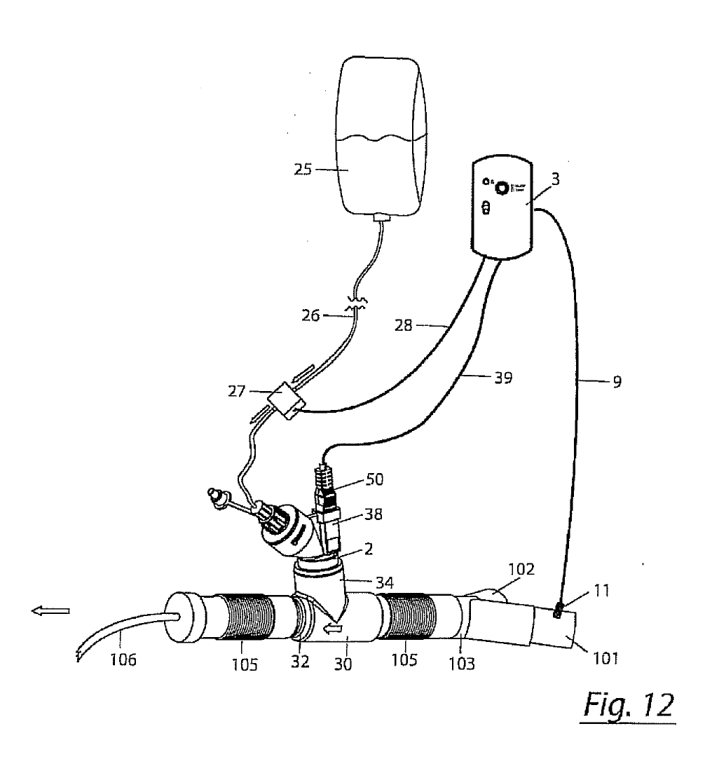

For non boosting applications such as active humidification a HME unit is not required and an aerosol generator 2 is placed between the wye junction 103 and the endotracheal tube 106 as illustrated in FIG. 12. In the arrangement of FIG. 12 100% humidity at the end of the ET tube 106 is achieved because all the humidity for the patient is provided by the nebuliser 2 with no passive humidification.

Aerosol can be delivered continuously, intermittently in short bursts or generated only on inspiration. A flow meter (sensor) 11 may be placed in the inspiratory tubing 101 so that the aerosol output can be adjusted to the inspiratory flow. This provides feedback to the controller 3 to provide aerosol while the sensor 11 detects flow to the patient which occurs in the inhaled breath. Sterile water or sterile normal saline is used as the humidifying agent and the system is sealed from the atmosphere reducing contamination risk.

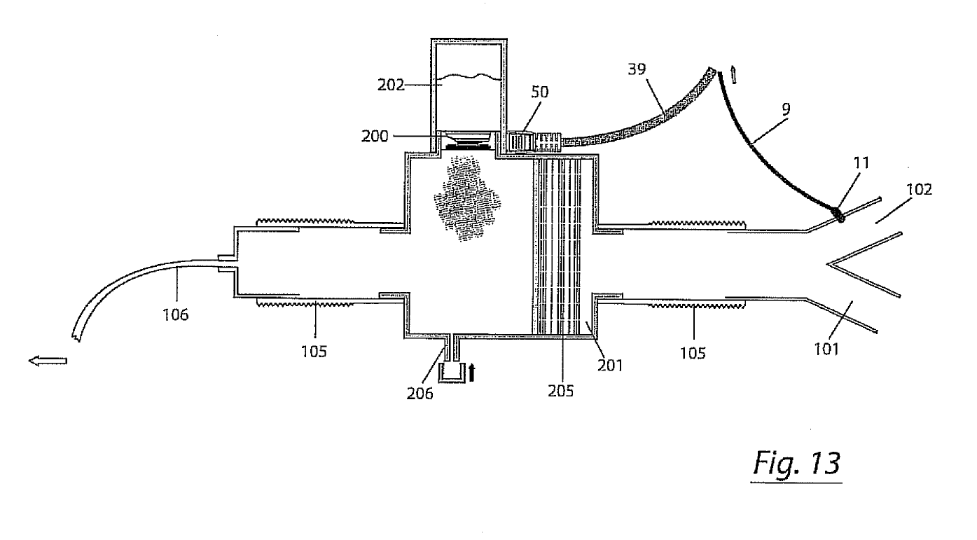

Another variant is illustrated in FIG. 13. This shows a combination of an aerosol generator 200 and a HME (Heat and Moisture Exchange) filter 201 that has an inbuilt liquid reservoir 202. This functions as a booster for passive humidification. In the arrangement of FIG. 13 100% humidity at the end of the ET tube is achieved by providing a top-up (boost) in humidity when using passive humidification via the HME, which is provided by an in-built aerosol generator 200 comprising a vibratable aperture plate which is incorporated into the HME. The HME unit 201 has a hydrophobic membrane 205 and is provided with a drain 206.

Referring to FIG. 1 (b) aqueous solution may be stored in the reservoir 1 of the nebuliser or the aqueous solution may be delivered to the reservoir 1 of the aerosol generator 2 in this case from a supply reservoir 25 along a delivery line 26. In the invention the flow of aqueous solution is assisted by an in-line flow controlling device 27 such as a pump and/or a valve which may be positioned in the delivery line 26. The operation of the flow controlling device 27 may be controlled by the controller 3 along a control wire 28 to ensure that the aerosol generator 2 has a supply of aqueous solution during operation and yet does not allow fluid build up which may affect the operation of the aersoliser. The device 27 may be of any suitable type.

The apparatus comprises a connector 30, in this case a T-piece connector 30 having a ventilation gas conduit inlet 31 and an outlet 32. The connector 30 also comprises an aerosol supply conduit 34 for delivering the aerosol from the aerosol generator 2 into the gas conduit 105 to entrain the aerosol with the ventilation gas, passing through the gas conduit 105. The entrained aerosol/ventilation gas mixture passes out of the connector 30 through the outlet 32 and is delivered to the endotracheal tube 106.

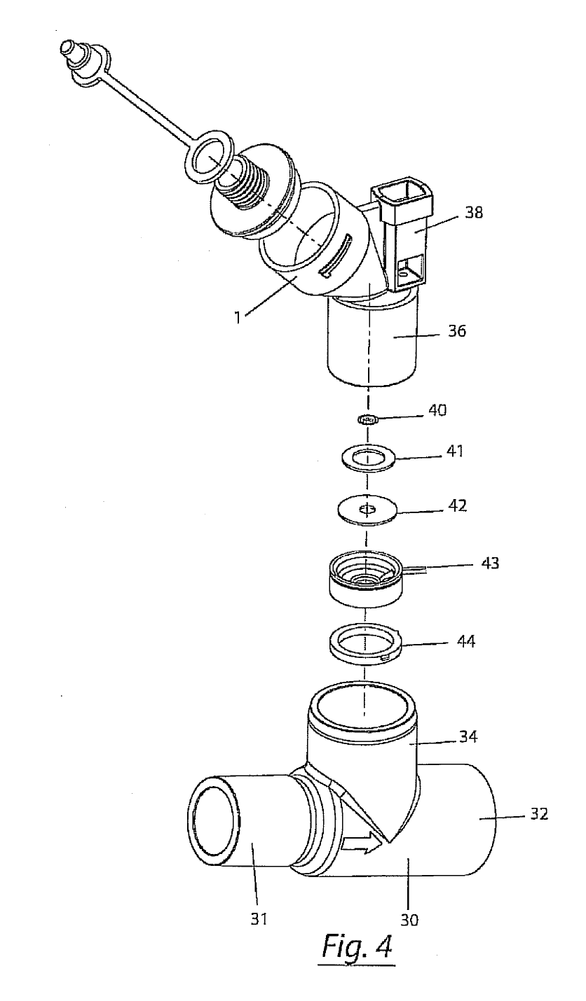

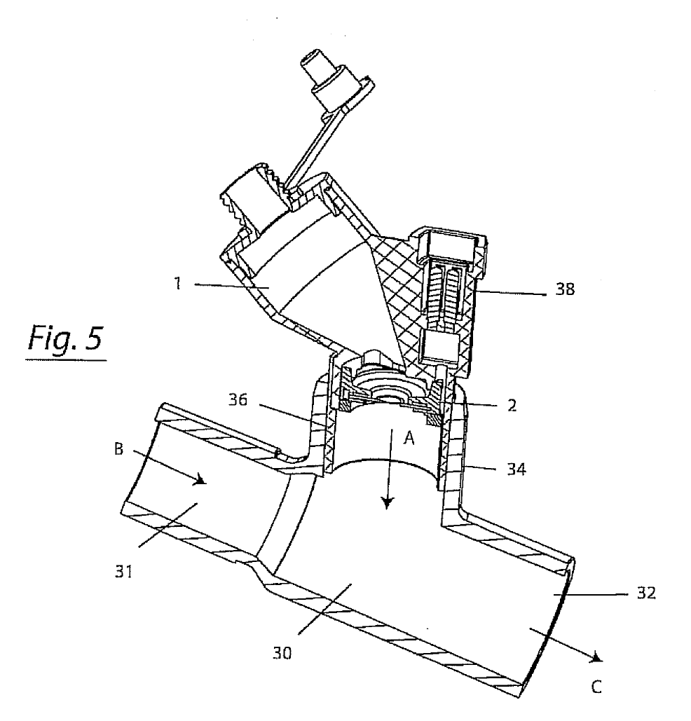

The aerosol supply conduit 34 and the ventilation gas conduit 105 meet at a junction. Referring particularly to FIGS. 4 and 5, in the assembled apparatus the aerosol supply conduit of the connector 30 may be releasably mounted to a neck 36 of the aerosol generator housing by means of a push-fit arrangement. This enables the connector 30 to be easily dismounted from the aerosol generator housing 36, for example for cleaning. The neck 36 at least partially lines the interior of the aerosol supply conduit 34.

The nebuliser (or aerosol generator) 2, has a vibratable member which is vibrated at ultrasonic frequencies to produce liquid droplets. Some specific, non-limiting examples of technologies for producing fine liquid droplets is by supplying liquid to an aperture plate having a plurality of tapered apertures extending between a first surface and a second surface thereof and vibrating the aperture plate to eject liquid droplets through the apertures. Such technologies are described generally in U.S. Pat. Nos. 5,164,740; 5,938,117; 5,586,550; 5,758,637; 6,014,970, 6,085,740, and US2005/021766A, the complete disclosures of which are incorporated herein by reference. However, it should be appreciated that the present invention is not limited for use only with such devices.

In use, the liquid to be aerosolised is received at the first surface, and the aerosol generator 2 generates the aerosolised liquid at the second surface by ejecting droplets of the liquid upon vibration of the vibratable member. The apertures in the vibratable member are sized to aerosolise the liquid by ejecting droplets of the liquid such that the majority of the droplets by mass have a size of less than 5 micrometers.

Referring particularly to FIGS. 4 and 5, in one case the aerosol generator 2 comprises a vibratable member 40, a piezoelectric element 41 and a washer 42, which are sealed within a silicone overmould 43 and secured in place within the housing 36 using a retaining ring 44. The vibratable member 40 has a plurality of tapered apertures extending between a first surface and a second surface thereof.

The first surface of the vibratable member 40, which in use faces upwardly, receives the liquid from the reservoir 1 and the aerosolised liquid, is generated at the second surface of the vibratable member 40 by ejecting droplets of liquid upon vibration of the member 40. In use the second surface faces downwardly. In one case, the apertures in the vibratable member 40 may be sized to produce an aerosol in which the majority of the droplets by weight have a size of less than 5 micrometers.

The vibratable member 40 could be non-planar, and may be dome-shaped in geometry.

The complete nebuliser may be supplied in sterile form, which is a significant advantage for use in breathing circuits.

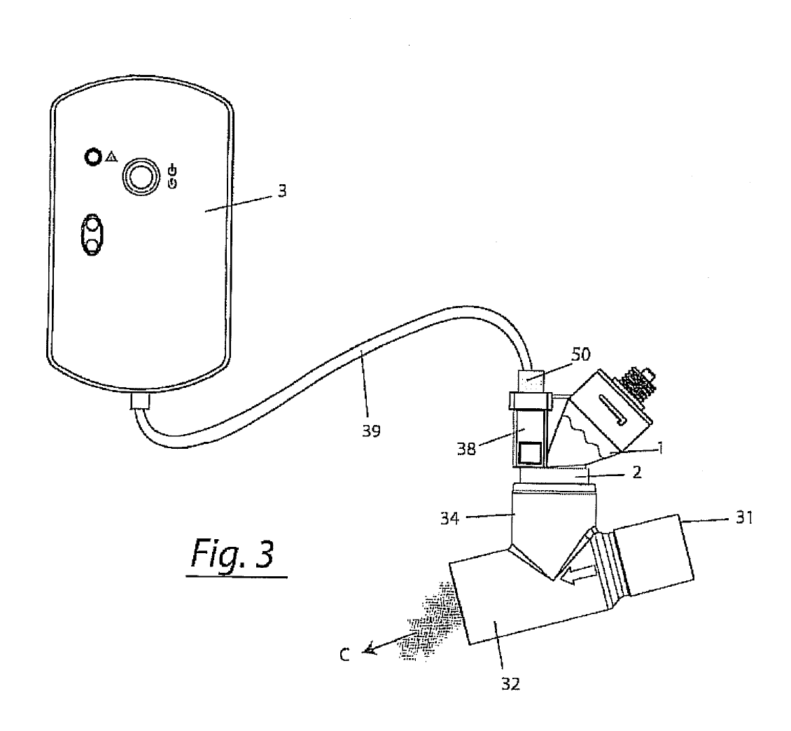

Referring particularly to FIG. 3, the controller 3 controls operation of and provides a power supply to the aerosol generator 2. The aerosol generator has a housing which defines the reservoir 1. The housing has a signal interface port 38 fixed to the lower portion of the reservoir 1 to receive a control signal from the controller 3. The controller 3 may be connected to the signal interface port 38 by means of a control lead 39 which has a docking member 50 for mating with the port 38. A control signal and power may be passed from the controller 3 through the lead 39 and the port 38 to the aerosol generator 2 to control the operation of the aerosol generator 2 and to supply power to the aerosol generator 2 respectively.

The power source for the controller 3 may be an on-board power source, such as a rechargeable battery, or a remote power source, such as a mains power source, or an insufflator power source. When the remote power source is an AC mains power source, an AC-DC converter may be connected between the AC power source and the controller 3. A power connection lead may be provided to connect a power socket of the controller 3 with the remote power source.

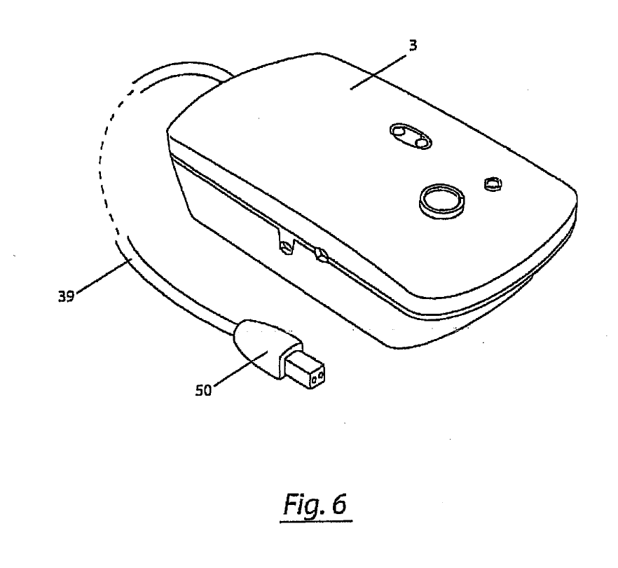

Referring particularly to FIG. 6 the controller 3 has a housing and a user interface to selectively control operation of the aerosol generator 2. Preferably the user interface is provided on the housing which, in use, is located remote from the aerosol generator housing. The user interface may be in the form of, for example, an on-off button. In one embodiment a button can be used to select pre-set values for simplicity of use. In another embodiment a dial mechanism can be used to select from a range of values from 0-100%.

Status indication means are also provided on the housing to indicate the operational state of the aerosol generator 2. For example, the status indication means may be in the form of two visible LED's, with one LED being used to indicate power and the other LED being used to indicate aerosol delivery. Alternatively one LED may be used to indicate an operational state of the aerosol generator 2, and the other LED may be used to indicate a rest state of the aerosol generator. 2.

A fault indicator may also be provided in the form of an LED on the housing. A battery charge indicator in the form of an LED may be provided at the side of the housing.

Referring particularly to FIGS. 1A and 1B, the liquid in the reservoir 1 flows by gravitational action towards the aerosol generator 2 at the lower medicament outlet. The controller 3 may then be activated to supply power and a control signal to the aerosol generator 2, which causes the piezoelectric element 41 to vibrate the non-planar member 40. This vibration of the non-planar member 40, causes the aqueous solution at the top surface of the member 40 to pass through the apertures to the lower surface where the aqueous solution is aerosolised by the ejection of small droplets of solution.

Referring particularly to FIGS. 4 and 5, the aerosol passes from the aerosol generator 2 into the neck 36 of the aerosol generator housing, which is mounted within the aerosol supply conduit of the connector 30 and into the gas conduit of the connector 30 (flow A). The aerosol is entrained in the ventilation gas conduit with gas, which passes into the gas conduit through the inlet 31 (flow B). The entrained mixture of the aerosol and the ventilation gas then passes out of the gas conduit through the outlet 32 (flow C) and on to the endotrachael tube 106.

The flow rate sensor/meter 11 determines the flow rate of the ventilation gas. In response to the fluid flow rate of the ventilation gas, the controller 3 commences operation of the aerosol generator 2 to aerosolise the aqueous solution. The aerosolised aqueous solution is entrained with the ventilation gas, and delivered to the patient.

In the event of alteration of the fluid flow rate of the ventilation gas, the flow rate sensor/meter 11 determines the alteration, and the controller 3 alters the pulse rate of the vibratable member of the nebuliser accordingly.

The controller 3 is in communication with the flow rate sensor/meter 11. The controller 3 is configured to control operation of the aerosol generator 2, responsive to the fluid flow rate of the ventilation gas and also independent of the fluid flow rate of the ventilation gas as required.

In one case, the controller 3 is configured to control operation of the aerosol generator 2 by controlling the pulse rate at a set frequency of vibration of the vibratable member, and thus controlling the fluid flow rate of the aqueous solutions.

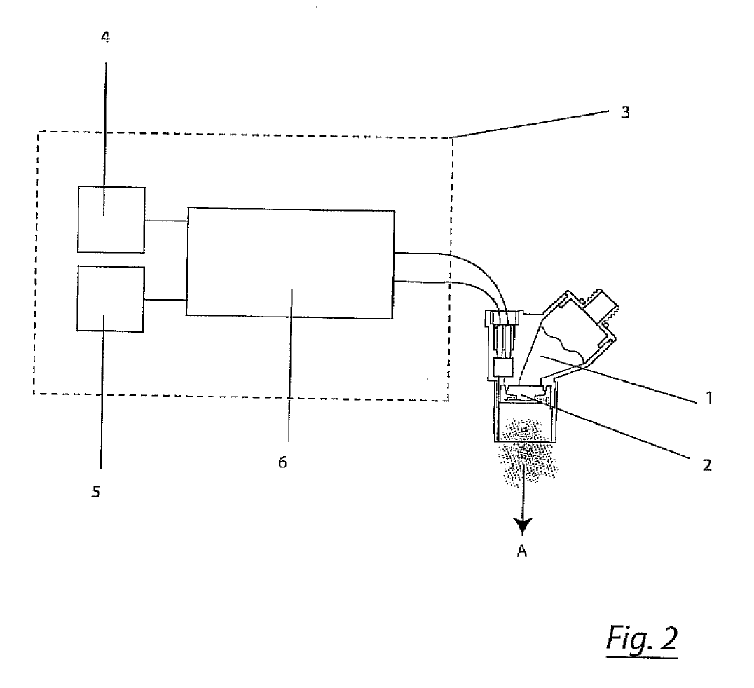

The controller 3 may comprise a microprocessor 4, a boost circuit 5, and a drive circuit 6. FIG. 2 illustrates the microprocessor 4, the boost circuit 5, the drive circuit 6 comprising impedance matching components (inductor), the nebuliser 2, and the aerosol. The inductor impedance is matched to the impedance of the piezoelectric element of the aerosol generator 2. The microprocessor 4 generates a square waveform of 128 KHz which is sent to the drive circuit 6. The boost circuit 5 generates a 12V DC voltage required by the drive circuit 6 from an input of either a 4.5V battery or a 9V AC/DC adapter. The circuit is matched to the impedance of the piezo ceramic element to ensure enhanced energy transfer. A drive frequency of 128 KHz is generated to drive the nebuliser at close to its resonant frequency so that enough amplitude is generated to break off droplets and produce the aerosol. If this frequency is chopped at a lower frequency such that aerosol is generated for a short time and then stopped for a short time this gives good control of the nebuliser's flow rate. This lower frequency is called the pulse rate.

The drive frequency may be started and stopped as required using the microprocessor 4. This allows for control of flow rate by driving the nebuliser 2 for any required pulse rate. The microprocessor 4 may control the on and off times to an accuracy of milliseconds.

The nebuliser 2 may be calibrated at a certain pulse rate by measuring how long it takes to deliver a know quantity of solution. There is a linear relationship between the pulse rate and the nebuliser flow rate. This may allow for accurate control over the delivery rate of the aqueous solution.

The nebuliser drive circuit consists of the electronic components designed to generate output sine waveform of approximately 100V AC which is fed to nebuliser 2 causing aerosol to be generated. The nebuliser drive circuit 6 uses inputs from microprocessor 4 and boost circuit 5 to achieve its output. The circuit is matched to the impedance of the piezo ceramic element to ensure good energy transfer.

The aerosol generator 2 may be configured to operate in a variety of different modes, such as continuous, and/or phasic, and/or optimised.

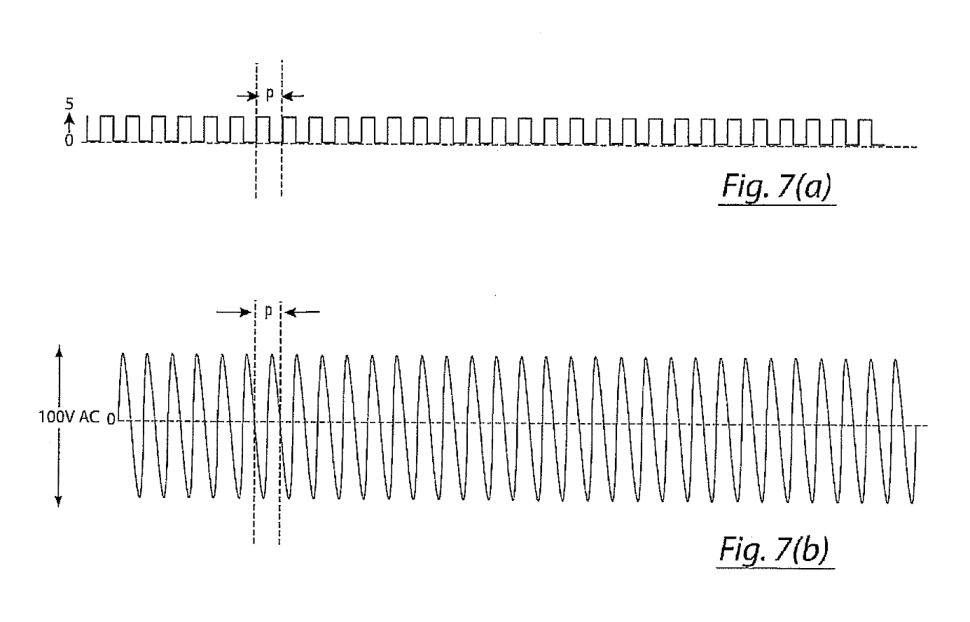

For example, referring to FIG. 7(a) illustrates a 5V DC square waveform output from the microprocessor 4 to the drive circuit 6. FIG. 7(b) shows a low power, ˜100V AC sine waveform output from drive circuit 6 to nebuliser 2. Both waveforms have a period p of 7.8 μS giving them a frequency of 1/7.8 μs which is approximately 128 KHz. Both waveforms are continuous without any pulsing. The aerosol generator may be operated in this mode to achieve 100% aerosol output.

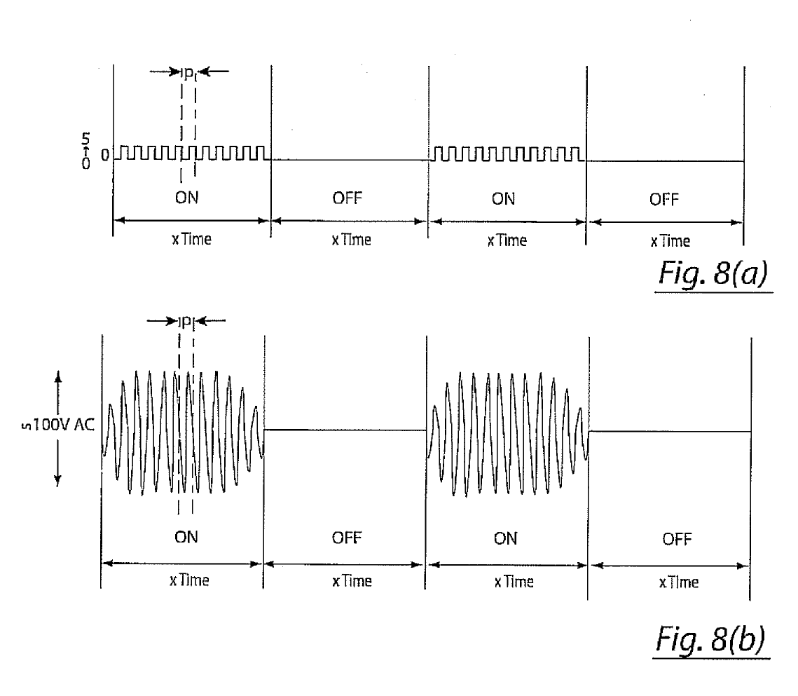

Referring to FIGS. 8(a) in another example, there is illustrated a 5V DC square waveform output from the microprocessor 4 to the drive circuit 6. FIG. 8(b) shows a low power, ˜100V AC sine waveform output from the drive circuit 6 to the nebuliser 2. Both waveforms have a period p of 7.8 μS giving them a frequency of 1/7.8 μs which is approximately 128 KHz. In both cases the wavefoms are chopped (stopped/OFF) for a period of time x. In this case the off time x is equal to the on time x. The aerosol generator may be operated in this mode to achieve 50% aerosol output.

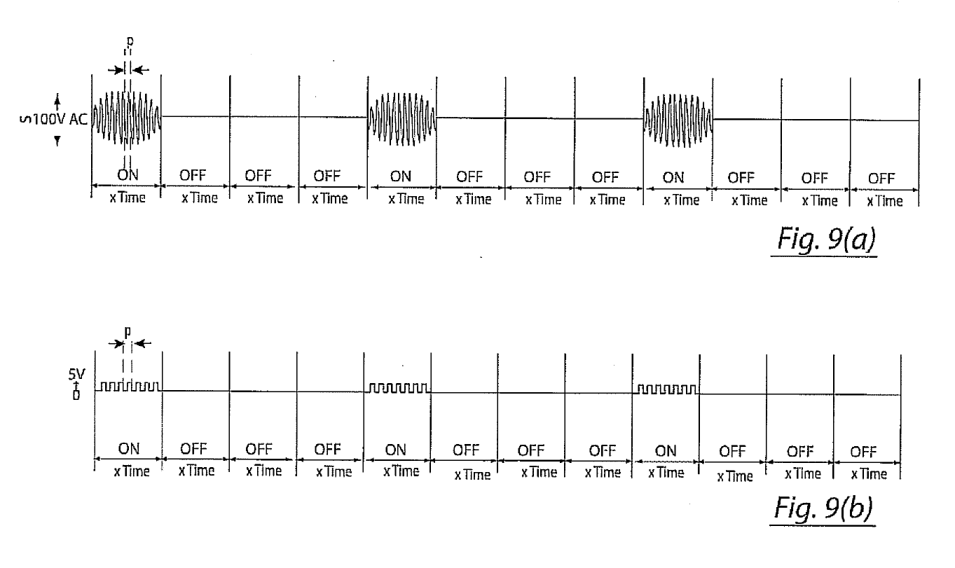

In another case, referring to FIGS. 9(a) there is illustrated a 5V DC square waveform output from microprocessor 4 to drive circuit 6. FIG. 9(b) shows a low power, ˜100V AC sine waveform output from the drive circuit 6 to the nebuliser 2. Both waveforms have a period p of 7.8 μS giving them a frequency of 1/7.8 μs which is approximately 128 KHz. In both cases the wavefoms are chopped (stopped/OFF) for a period of time x. In this case the off time is 3× while the on time is x. The aerosol generator may be operated in this mode to achieve 25% aerosol output.

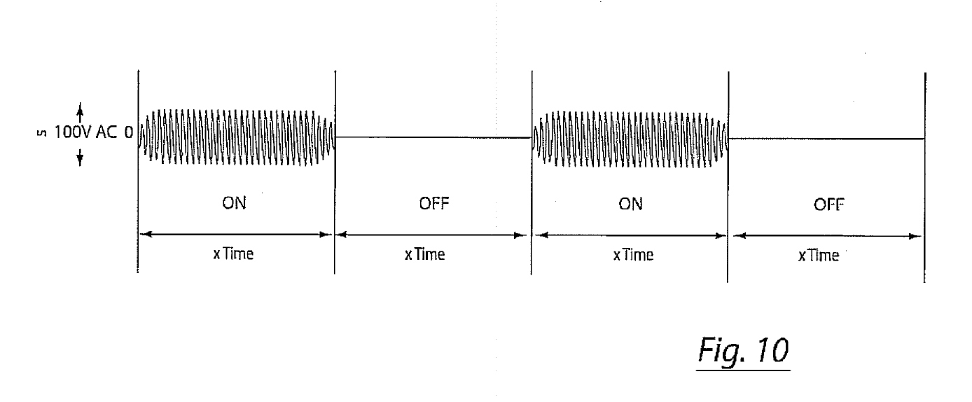

Referring to FIG. 10, in one application pulsing is achieved by specifying an on-time and off-time for the vibration of the aperture plate. If the on-time is set to 200 vibrations and off-time is set to 200 vibrations, the pulse rate is 50% (½ on ½ off). This means that the flow rate is half of that of a fully driven aperture plate. Any number of vibrations can be specified but to achieve a linear relationship between flow rate and pulse rate a minimum number of on-time vibrations is specified since it takes a finite amount of time for the aperture plate to reach its maximum amplitude of vibrations.

The drive frequency can be started and stopped as required by the microprocessor; this allows control of flow rate by driving the nebuliser for any required pulse rate. The microprocessor can control the on and off times with an accuracy of microseconds.

A nebuliser can be calibrated at a certain pulse rate by measuring how long it takes to deliver a known quantity of solution. There is a linear relationship between the pulse rate and that nebuliser's flow rate. This allows accurate control of the rate of delivery of the aerosolised aqueous solution.

The pulse rate may be lowered so that the velocity of the emerging aerosol is much reduced so that impaction rain-out is reduced.

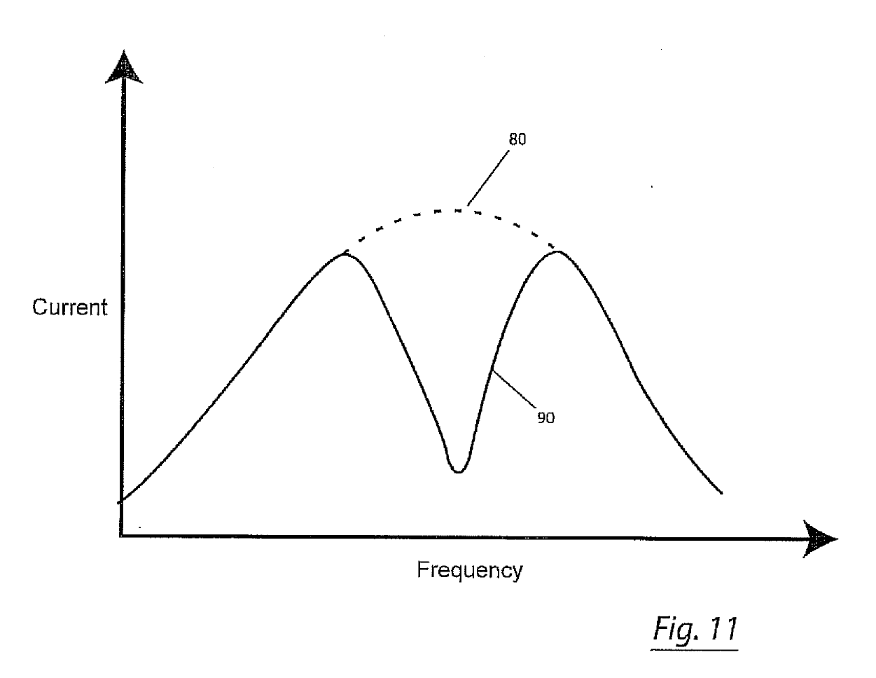

Detection of when the aperture plate is dry can be achieved by using the fact that a dry aperture plate has a well defined resonant frequency. If the drive frequency is swept from 120 kHz to 145 kHz and the current is measured then if a minimum current is detected less than a set value, the aperture plate must have gone dry. A wet aperture plate has no resonant frequency. The apparatus of the invention may be configured to determine whether there is any of the first fluid in contact with the aerosol generator 2. By determining an electrical characteristic of the aerosol generator 2, for example the current flowing through the aerosol generator 2, over a range of vibration frequencies, and comparing this electrical characteristic against a pre-defined set of data, it is possible to determine whether the aerosol generator 2 has any solution in contact with the aerosol generator 2. FIG. 11 illustrates a curve 80 of frequency versus current when there is some of the solution in contact with the aerosol generator 2, and illustrates a curve 90 of frequency versus current when there is none of the solution in contact with the aerosol generator 2. FIG. 11 illustrates the wet aperture plate curve 80 and the dry aperture plate curve 90.

If an application requires a constant feed from a drip bag then a pump can be added in line to give fine control of the liquid delivery rate which can be nebulised drip by drip. The rate would be set so that liquid would not build up in the nebuliser. This system is particularly suitable for constant low dose delivery.

In the invention the aerosol generator is placed at the patient's endotracheal tube so there is little to no rain-out in the tubing.

The device is very light and unlike the full heated wire system and very silent unlike the jet nebulizer. Non-heated single patient use ventilator tubing can be used. These bring considerable benefits:
reduced cost of maintenance (care giver time)
reduced heat/power cost (in excess of 10 fold)
reduced cost of capital equipment and circuits
reduced background noise

The supply to the aerosol generator is sealed from the atmosphere as it is in a closed circuit and so minimises infection risk even though the aerosol particle size is large enough to carry bacteria.

Intermittent short bursts of aerosol can be programmed to optimize water and heat replenishment of the HME, without requiring more complex aerosol generation patterns.

A drip feed line fed into a small volume reservoir allows the nebuliser to work in almost any orientation, reducing work and risk for the care giver. This also can provide a very low weight, low profile device.

With the aerosol used to augment the HME, another nebulizer for medication delivery can be placed between the HME and the patient ET, as described for example in US2005/0139211A, the entire contents of which are incorporated herein by reference.

The invention can be applied to systems used to ventilate all patients requiring mechanical ventilation or having bypassed upper airways requiring supplemental humidification.

All patients on mechanical ventilation require humidification either with a heated wire humidifier or a heat moisture exchanger. This system can be configured to add humidity and heat to a heat moisture exchange system thereby increasing the ability of patient with thick secretions to be adequately humidified with a HME only system or to fully replace a heated wire humidifier system by adding sufficient amounts of aerosol to the inspired air.

A major problem with the use of nebulizers in the past was contamination of the patient. This has generally been ascribed to the fact that the aerosol particles are of sufficient size to carry bacteria whereas vapour particles are not. The fact that the Aerogen nebulizers can be sterilized and also have the capacity to have a continuous feed of sterile liquid will overcome this reported disadvantage.

The key advantageous features of the invention are:
small/compact
quiet
fed from a sterile sealed system
no heated wire tubing
no large power supply
lower cost
position independent operation

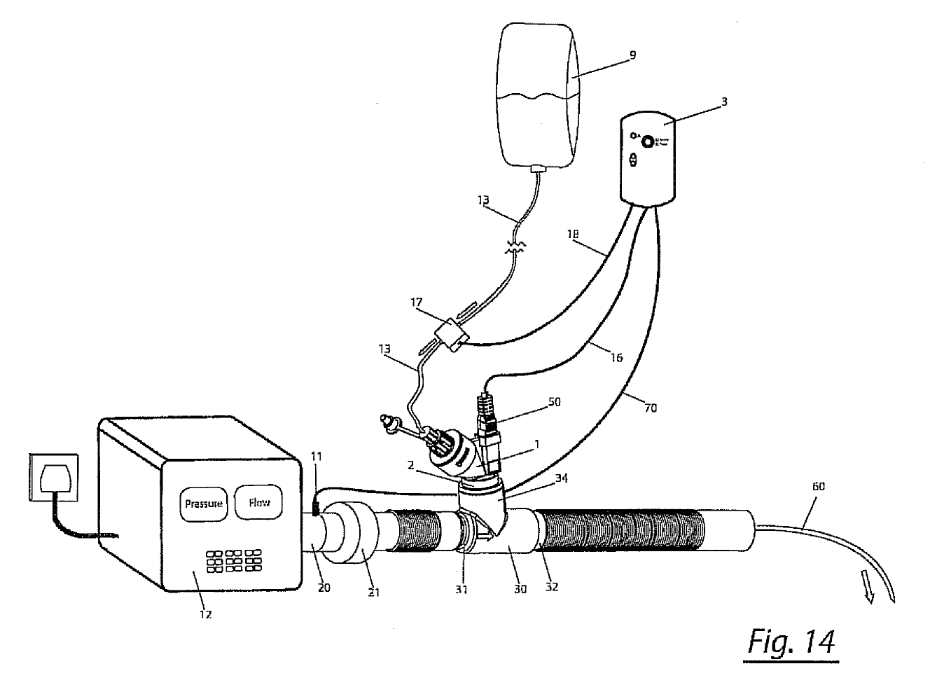

Referring to FIG. 14 there is illustrated an apparatus according to the invention for use in insufflation of a body cavity. One such application is laparoscopic surgery. The device is also suitable for use in any situation involving insufflation of a body cavity such as in arthroscopies, pleural cavity insufflation (for example during thoracoscopy), retroperitoneal insufflations (for example retroperitoneoscopy), during hernia repair, during mediastinoscopy and any other such procedure involving insufflation.

The apparatus comprises a reservoir 1 for storing an aqueous solution, an aerosol generator 2 for aerosolising the solution, and a controller 3 for controlling operation of the aerosol generator 2. The aqueous solution is fed from a reservoir 9 to the aerosol generator 2 along a delivery tube 13. In the invention aerosolised aqueous solution is entrained with insufflation gas. The gas is any suitable insufflation gas such as carbon dioxide. Other examples of suitable insufflation gases are nitrogen, helium and xenon.

The insufflation gas is delivered into an insufflation gas tubing 15 by an insufflator 12. The insufflator 12 may be of any suitable type such as those available from Karl Storz, Olympus and Stryker. The insufflator 12 has an outlet 20 through which insufflation gas is delivered. A bacterial filter 21 may be provided within the insufflator or, as illustrated, downstream of the insufflator outlet 20.

In this case a flow rate sensor/meter 11 is located in the flow path of the insufflation gas from an insufflator 12 to the aerosol generator 2. The flow rate sensor/meter 11 is connected by a control wire 70 to the controller 3, and the aerosol generator 2 is connected to the controller 3 by a control wire 16. The flow rate sensor/meter 11 may be a hot wire anemometer, or in the case where the flow is laminar or can be laminarised, a differential pressure transducer.

Sterile water may be used. In the case of an aqueous solution any suitable solution may be used. Solutions with a salt concentration in the range 1 μM (micro molar) to 154 mM (milli molar) (0.9% saline) are optimum as they cover the majority of medical applications. In addition, such saline concentrations can be readily nebulised using the aerosolisation technology used in the invention.

Aqueous solution may be stored in the reservoir 1 container of the nebuliser or the aqueous solution may be delivered to the reservoir 1 of the aerosol generator 2 in this case from the supply reservoir 9 along the delivery line 13. The flow of aqueous solution may be by gravity and/or may be assisted by an in-line flow controlling device 17 such as a pump and/or a valve which may be positioned in the delivery line 13. The operation of the flow controlling device 17 may be controlled by the controller 3 along a control wire 18 to ensure that the aerosol generator 2 has a supply of aqueous solution during operation. The device 17 may be of any suitable type.

The apparatus comprises a connector 30, in this case a T-piece connector 30 having an insufflation gas conduit inlet 31 and an outlet 32. The connector 30 also comprises an aerosol supply conduit 34 for delivering the aerosol from the aerosol generator 2 into the insufflation gas conduit 15 to entrain the aerosol with the insufflation gas, passing through the gas insufflation conduit 15. The entrained aerosol/insufflation gas mixture passes out of the connector 30 through the outlet 32 and is delivered to the body cavity along a line 60.

In use during laparoscopic surgery the flow of the insufflation gas into the abdomen of a patient is commenced to insufflate the abdomen. The flow rate sensor/meter 11 determines the flow rate of the insufflation gas. In response to the fluid flow rate of the insufflation gas, the controller 3 commences operation of the aerosol generator 2 to aerosolise the aqueous solution. The aerosolised aqueous solution is entrained with the insufflation gas, and delivered into the abdomen of the patient to insufflate at least part of the abdomen.

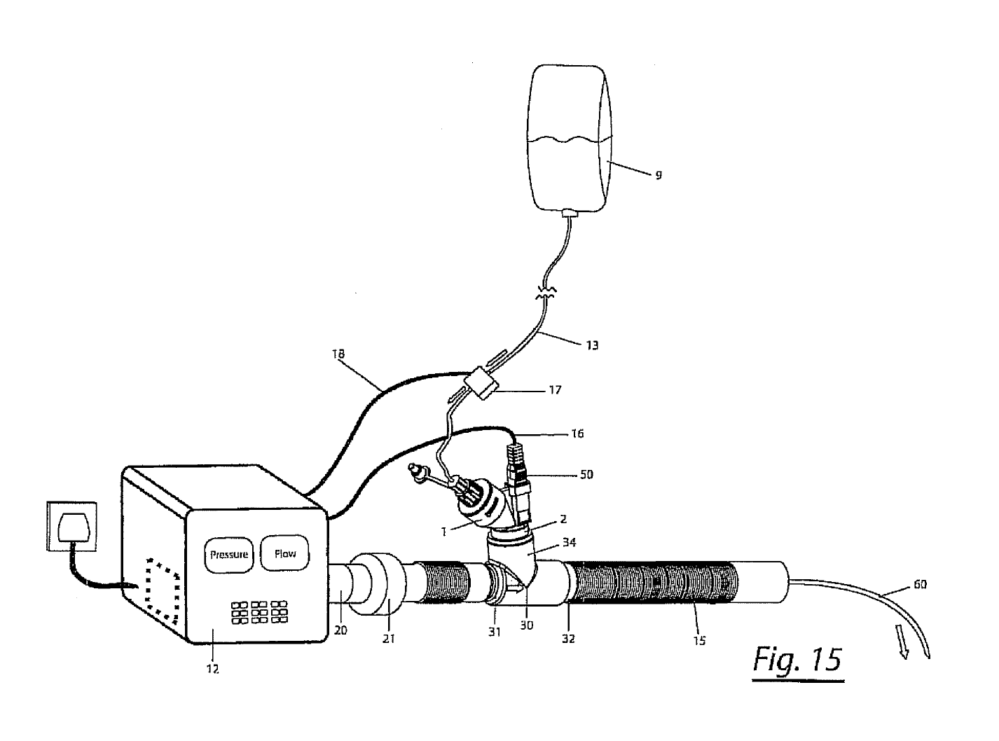

If an application requires a constant feed from a drip bag then a pump can be added in line to give fine control of the liquid delivery rate which can be nebulised drip by drip. The rate would be set so that liquid would not build up in the nebuliser. This system is particularly suitable for constant low dose delivery. Referring now to FIG. 15 there is illustrated another insufflation apparatus which is similar to the apparatus of FIG. 1 and like parts are arranged the same reference numerals. In this case the controller 3 is integrated into the insufflator 12. The insufflator 12 would have information on the rate of flow that it is producing and using an integrated circuit board may directly communicate with the nebuliser 2. This would eliminate the need for the separate flowmeter 11 and the stand-alone controller 3 to be present.

In another case there may be a common information bus between the insufflator 12 and the controller 3. The insufflator 12 would have information on the rate of flow that it is producing and would communicate this to the controller 3 and on to the nebuliser 2, thereby eliminating the need for the flowmeter 11. This would allow the invention to be backward compatible with a variety of types of insufflator.

Referring to FIG. 15 there is illustrated another insufflation apparatus which is similar to the apparatus of FIG. 14 and like parts are again identified by the same reference numerals. In this case the insufflation gas flow signal is provided directly from the insufflator along a lead 71. One advantage of this arrangement is that no separate meter/sensor required.

Humidity may be generated via the aerosolisation of any aqueous solution. Relative humidity in the 50-100% range would be optimum. The control module can generate a nebuliser output of any defined relative humidity percentage based on the insufflator flow. These solutions include any aqueous drug solution. Solutions with salt concentrations in the range 1 μM-154 mM would be optimum.

The use of the nebulizer to humidify the insufflation gas prior to entering the body will eliminate the need for the body to humidify the gas once it is inside the body, thereby minimizing body heat loss by internal evaporation.

The control in nebulizer output allows proportional delivery of the required amount of humidity according to the amount of insufflation gas entering the body. In addition this control of aerosolization rate will prevent overloading of the insufflation gas with aerosol which would obscure the surgeons view.

In addition to acting as a humidifying agent the nebulizer can also act to deliver any agent presented in an aqueous drug solution. The system facilitates delivery of, for example, pain-relief medications, anti-infectives, anti-inflammatory and/or chemotherapy agents in aerosol form to the body cavity. These therapeutic agents could also act as humidifying substances in their own right.

The liquid entrained in the insufflation gas may contain any desired therapeutic and/or prophylactic agent. Such an agent may for example be one or more of an analgesic, an anti-inflammatory, an anaesthetic, an anti-infective such as an antibiotic, or an anti-cancer chemotherapy agent.

Typical local anaesthetics are, for example, Ropivacaine, Bupivacaine and Lidocaine. Typical anti-infectives include antibiotics such as an aminoglycoside, a tetracycline, a fluoroquinolone; anti-microbials such as a cephalosporin; and anti-fungals.

Anti-inflammatories may be of the steroidal or non-steroidal type.

Anti-cancer chemotherapy agents may be alkylating agents, antimetabolites anthracyclines, plant alkaloids, topoisomerase inhibitors, nitrosoureas, mitotic inhibitors, monoclonal antibodies, tyrosine kinase inhibitors, hormone therapies including corticosteroids, cancer vaccines, anti-estrogens, aromatase inhibitors, anti-androgens, anti-angiogenic agents and other antitumour agents.

The system of the invention can be used for precise controlled delivery of drug and/or humidity during insufflation. No heating is required. Consequently there is no risk of damage to drugs due to heating. The system may be used to provide precise control over aerosol output can be exercised by utilising pulse rate control. The system may be used for targeted delivery of a range of drugs, thereby reducing systemic side effects. In addition the system provides alleviation of post-surgical pain experienced by the patient.

The system need not be located in the direct flow path of insufflation gas. In addition, minimal caregiver intervention during laparoscopic procedure is required. The system is small and compact and allows for integration with an insufflator.

The device of the invention can be used throughout the procedure carried out by a surgeon. The device ensures that humidity is actively controlled during the procedure and thus ensures that a surgeon's view is clear as fogging is avoided.

All parts of the device (except the controller and associated leads) are autoclavable which provides a significant advantage for a device used in surgery.

The invention solves a number of problems:
it allows for multi-orientation aerosol delivery;
it allows for finely controlled volume delivery output independent of the aerosol rate of the individual nebuliser;
it allows for a much greater range of liquid viscosities/types to be aerosolised using vibrating mesh technology; and/or
it eliminates the need for an infusion/peristaltic pump, which can be cumbersome and expensive.

The invention provides a compact low-cost solution to aerosolization or nebulization of liquids/drugs is required giving finer control than what was previously available. Such applications include but are not limited to continuous and intermittent drug delivery for respiratory and surgical applications; and or delivery of non-drug solutions and suspensions for aerosol equipment calibration.

The devices are small, provide precise fluid delivery, the pump may be integrated with the nebuliser. The device is not gravity dependant so that multiple orientation is possible.

The invention is not limited to the embodiments hereinbefore described which may be varied in detail.

1. An aerosolisation system comprising an aerosol generator and a flow controlling device for delivery of fluid to be aerosolised to the aerosol generator.

2. A system as claimed in claim 1 wherein the flow controlling device comprises a micropump.

3. A system as claimed in claim 2 wherein the micropump comprises a diaphragm pump.

4. A system as claimed in claim 3 wherein the diaphragm pump is driven by piezo activation.

5. A system as claimed in claim 1 wherein the flow controlling device comprises a microvalve.

6. A system as claimed in claim 5 wherein the valve is a solenoid valve.

7. A system as claimed in claim 1 wherein the aerosol generator comprises a vibratable member having a plurality of apertures extending between a first surface and a second surface thereof.

8. A system as claimed in claim 7 wherein the first surface is adapted to receive fluid to be aerosolised.

9. A system as claimed in claim 7 wherein the aerosol generator is configured to generate an aerosol at the second surface.

10. A system as claimed in claim 7 wherein the vibratable member is dome-shaped in geometry.

11. A system as claimed in claim 7 wherein the vibratable member comprises a piezoelectric element.

12. A system as claimed in claim 7 wherein the apertures in the vibratable member are sized to aerosolise fluid by ejecting droplets of the water such that the majority of the droplets by mass have a size of less than 5 micrometers.

13. A system as claimed in claim 7 wherein the apertures in the vibratable member are sized to aerosolize fluid by ejecting droplets of the water such that the majority of the droplets by mass have a size of less than 3 micrometers.

14. A system as claimed in claim 1 comprising a controller for controlling the operation of the aerosol generator and the micropump.

15. A system as claimed in claim 14 wherein the controller is configured to control the pulse rate at a set frequency of vibration of the vibratable member.

16. A system as claimed in claim 14 wherein the controller is impedance matched to the aerosol generator.

17. A system as claimed in claim 1 wherein the apparatus comprises means to determine whether fluid is in contact with the aerosol generator.

18. A system as claimed in claim 17 wherein the determining means is configured to determine at least one electrical characteristic of the aerosol generator.

19. A system as claimed in claim 17 wherein the determining means is configured to determine at least one electrical characteristic of the aerosol generator over a range of vibration frequencies.

20. A system as claimed in claim 17 wherein the determining means is configured to compare the at least one electrical characteristic against a pre-defined set of data.
